(19) United States
(12) Patent Application Publication
(10) Pub. No.: US 2007/0276508 A1
(43) Pub. Date: Nov. 29, 2007

(54) Artificial lung system and its use

(76) Inventors: Stefan Fischer, Roesrath (DE); Georg Matheis, Burladingen (DE)

Correspondence Address: MORRISON & FOERSTER LLP, 12531 HIGH BLUFF DRIVE, SUITE 100, SAN DIEGO, CA, 92130-2040, US

(21) Appl. No.: 11/788,579

(22) Filed: Apr. 20, 2007

(30) Foreign Application Priority Data
Apr. 21, 2006 (DE) 10 2006 020 494.8

(51) Int. Cl. A61F 2/04

(52) U.S. Cl. 623/23.65

(57) ABSTRACT

In an artificial lung system, a gas exchange membrane separates a blood side from an air side, the gas exchange membrane comprising in each case, on the blood side and on the air side, a foreign surface that is colonized on the blood side and/or on the air side with biological cells. The artificial lung system can be used to produce an extracorporeal or implantable lung assist system or to produce a lung model system for the examination of airway stresses.

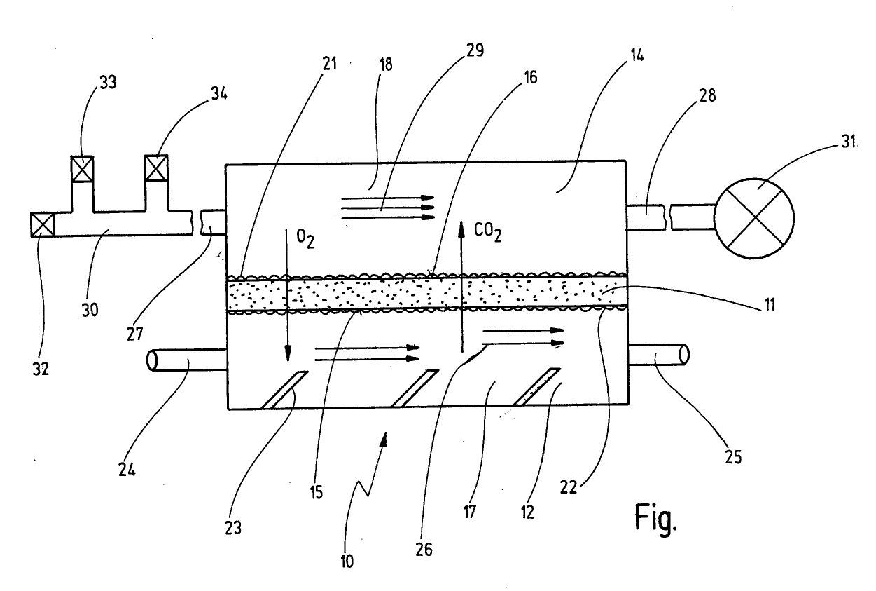

### CROSS-REFERENCE TO RELATED APPLICATIONS

This application claims priority under 35 U.S.C. § 119(d) from German Patent Application No. 10 2006 020 494.8, filed Apr. 21, 2006. The content of the above patent application is incorporated by reference herein in its entirety.

### BACKGROUND OF THE INVENTION

### Field of the Invention

The present invention relates to an artificial lung system with a gas exchange membrane that separates a blood side from an air side, the gas exchange membrane comprising a foreign surface both on the blood side and on the air side.

The invention further relates to the use of the artificial lung system for producing an extracorporeal or implantable lung assist system and for producing a lung model system as a replacement for animal model systems in toxicology studies.

An artificial lung system of the type mentioned at the outset, which is used as an extracorporeal lung assist system, is marketed by the Applicant under the trade name NovaLung iLA (interventional Lung Assist).

An overview of new technologies for lung assist systems is given by G. Matheis: “New technologies for respiratory assist” in Perfusion (2003) 18:245-251. Matheis describes, among other things, the aforementioned NovaLung iLA, which is based on a membrane lung and is designed for pulsatile blood flow with dense diffusion membranes that are coated with a nonvital protein matrix.

On the blood side, the NovaLung iLA is connected directly to the blood circulation of a patient by percutaneous arterial and venous cannulation. The NovaLung iLA makes it possible, without using a blood pump, to remove carbon dioxide from the blood being pumped from the patient's heart through the membrane lung and to oxygenate it under the limitations of the inflow of arterial blood.

The first successful application of a precursor of the NovaLung iLA as lung assist system is reported by Reng et al.: “Pumpless extracorporeal lung assist and adult respiratory distress syndrome” in The Lancet (2000) 356:219-220.

The importance of the NovaLung iLA, and of the artificial lung systems according to the invention, is to be seen against the fact that lung diseases are the third most common cause of death according to statistics from the World Health Organization. Although mechanical ventilation is able to maintain gas exchange in almost all patients, the unnatural positive airway pressure causes injury to the lungs and other organs, known as ventilator-associated lung injury (VALI). With the NovaLung iLA, it is possible for the first time, by means of ventilation outside the lung, to achieve a highly protective ventilation or, for example, a bridge to lung transplantation, and to avoid VALI.

At present, however, there is no organ replacement method available that can be used on a long-term basis for the lung, for example in the way that dialysis can be performed for serious kidney failure, nor is there a fully implantable system available that can completely replace the lung, for example in the manner of heart support systems. In cases of lung failure, therefore, the only possibility at present lies in mechanical ventilation. However, this does not constitute lung support, since the diseased lung is not treated. Instead, it ensures only the gas exchange necessary for life.

Provided that they satisfy strictly defined inclusion criteria, patients with seriously damaged lungs are therefore candidates for lung transplantation. Lung transplantation, however, represents an extremely complex medical procedure, and one that is also associated with a high risk to the patient. Besides the fact that this therapy concept is reserved exclusively for patients who have an isolated lung disease and are otherwise healthy, the long-term results are unsatisfactory. A further consideration is that, because of the small number of donor organs that become available each year across the world, only a small number of lung transplantation procedures can be performed, and this does not meet the actual demand.

Against this background, there is a significant need for a lung replacement procedure as a treatment that prolongs life (destination therapy). An extracorporeal or implantable lung assist system that can oxygenate the blood and can also remove carbon dioxide from the blood is therefore of very great value to patients. Such lung assist systems, however, can be used not only on patients who are unsuitable for a transplantation, but also on patients who are waiting for a lung transplantation and who, during the waiting period, develop critical lung failure that necessitates ventilation.

The fact that artificial lung assist systems such as the NovaLung iLA can be used in principle for this purpose has been shown in a clinical study—see Fischer et al.: BRIDGE TO LUNG TRANSPLANTATION WITH THE NOVEL PUMPLESS INTERVENTIONAL LUNG ASSIST DEVICE NOVALUNG, in J. Thorac. Cardiovasc. Surg. (2006) 131(3):719-723.

In the context of the above study, patients who developed lung failure that failed to respond to mechanical ventilation were successfully bridged to transplantation by means of the NovaLung iLA lung assist system. At present, such systems can be used in intensive care units for a period of a few weeks. Longer use of the NovaLung iLA is not possible because of neointima formation and other factors, unless the membrane lung is regularly replaced.

Against this background, it is an object of the present invention to develop the artificial lung system mentioned at the outset in such a way that it can be used not just for short-term lung support, but also for medium-term and long-term extracorporeal lung support or for producing an implantable lung assist system.

### SUMMARY OF THE INVENTION

According to the invention, this object is achieved, in terms of the artificial lung system mentioned at the outset, by the fact that the foreign surface on the blood side and/or the foreign surface on the air side is colonized with biological cells.

This object of the invention is achieved in full by this means.

The inventors of the present application have in fact found that the neointima formation and the activation of inflammatory reactions is associated with the fact that the known lung assist systems have foreign surfaces that come into contact permanently with the blood. Although the foreign surfaces of the known lung assist systems are provided with a nonvital coating of protein and heparin, systemic reactions (pro-inflammatory immune response) occur even after short-term use, as are also known from other clinical applications of organ support systems with foreign surfaces that come into contact with blood. Examples of such organ support systems are heart-lung machines with an oxygenator, mechanical blood pumps, haemodialysis and heart support systems, for example artificial hearts. In addition to the contact between blood and the foreign surface, mechanical blood pumps, which are necessary in all known organ support systems, cause unphysiological shearing forces, with corresponding damage to the blood.

These problems were able to be overcome using the NovaLung iLA artificial lung perfused with the patient's blood, because blood is circulated through the system physiologically by the patient's heart, but the known system cannot be used on a long-term basis.

According to the invention, a lung assist system that can be used on a long-term basis is colonized with cells in order to be able to function successfully, these cells being provided with physiological perfusion and ventilation conditions. These requirements are now completely satisfied by the artificial lung system according to the invention.

According to the invention, a kind of biohybrid lung is thus made available that serves to replace or support the lung function. This affords the advantage that individualized, cellularized surfaces replace the surfaces conventionally provided with a nonvital coating.

In this way, it is possible to produce an artificial lung which can be used short-term or long-term and which is able to completely replace the gas exchange function of a diseased lung, and which therefore can sufficiently oxygenate the blood and at the same time can also remove carbon dioxide from the blood.

It is advantageous if the foreign surfaces are colonized completely with biological cells, preferably autologous cells, in order to avoid a foreign surface on the blood side and/or on the air side. For this purpose, the blood side is colonized with endothelial cells and the air side is colonized with alveolar epithelial cells (pneumocytes).

It is not absolutely essential for both the blood side and also the air side to be colonized with biological cells. The respective other side can also be provided with a nonvital coating, for example with protein plus heparin.

If only the blood side is colonized with suitable endothelial cells, and the foreign surface there is therefore covered completely by a cell lawn, the inflammatory reactions known in the prior art no longer occur, such that, for this reason alone, the artificial lung system according to the invention has a much longer useful life in a patient's blood flow than do the known lung assist systems.

If, by contrast, the air side is colonized with pneumocytes, a kind of biological defense of the lung assist system takes place, with the pneumocytes thus forming, as epithelium, the barrier to the individual. The epithelial cells are supplied via the blood stream, that is to say through the gas exchange membrane.

The biological cells can be, example given, stem cells, progenitors or differentiated cells. In particular embryonic stem cells, stem and progenitor cells from umbilical cord blood, adult mesenchymal stem cells, adult stem cells, endothelial progenitor cells or endothelial cells represent suitable sources for epithelial cells. As a source for pneumocytes, in particular embryonic stem cells, stem or progenitor cells from umbilical cord blood, adult mesenchymal stem cells, adult stem cells, pulmonary progenitor cells, differentiated alveolar epithelial cells, in particular of type I and II, are suited.

In particular, the biological cells can be taken from the respiratory tract (epithelial cells) or from a segment of a superficial cutaneous vein (endothelial cells) and then cultivated. However, initial studies by the Applicant have shown that the biological cells can also be cultured for example from umbilical cord cells.

Regarding the artificial lung system according to the invention, the gas exchange membrane represents a form of a separation layer, which is manufactured of either a natural or an artificial material, or mixtures there from. In addition, the materials to be employed can be biodegradable materials.

According to the invention, the blood side of the artificial lung system comprises a closed blood chamber with inlet and outlet ports for attachment to a natural blood circuit or to an artificial perfusion system, the air side preferably comprising a closed air chamber with inlet and outlet ports for attachment to natural airways or to an artificial ventilation system.

If the artificial lung system is used to produce an extracorporeal lung assist system, the blood-side blood chamber is attached by percutaneous cannulation or by subcutaneous vascular prostheses to an artery and a vein, for example to the subclavian artery and to the subclavian vein. On the air side, an artificial ventilation system is then provided which ventilates the air chamber physiologically, such that the pneumocytes are ventilated with an underpressure, in the same way as in natural inhalation.

If the artificial lung system is used to produce an implantable lung assist system, the air chamber by contrast is linked by an inlet port to the natural airways, for example to the trachea or bronchi. An inlet port of the air chamber of the artificial lung system is then designed for attachment to natural airways, and the other inlet port of the air chamber can be connected to a pressure chamber that generates an oscillating air stream in the air chamber by means of alternating expansion and compression.

However, it is also possible to use the artificial lung system to produce a lung model system for examining the toxicity of pharmaceuticals or for examination of airway stresses, for example caused by contaminants, such that it can serve as a replacement for animal model systems in toxicology studies. In this case, the blood side is attached to an artificial perfusion system, for example an artificial blood circuit, which is designed such that the endothelial cells are perfused physiologically, that is to say in a pulsatile manner. The air side is then attached to the artificial ventilation system, via which the pneumocytes are ventilated physiologically, that is to say with underpressure.

In addition, the artificial ventilation system then preferably comprises an inlet for foreign substances, such as volatile substances or foreign gases, whose effect on the pneumocytes and/or endothelial cells is to be tested.

It is not absolutely essential for human or animal blood to circulate in the artificial perfusion system. Instead, artificial blood can be used or some other suitable biological medium via which the biological cells are supplied with nutrients.

With this lung model system, the toxicological effect, for example of volatile substances or foreign gases, on the function of the pneumocytes and endothelial cells can be tested without having to use animal model systems for this purpose.

It is generally advantageous, in the artificial lung system, if the gas exchange membrane is a diffusion membrane made preferably from polymethylpentene (PMP), and the gas exchange membrane, or, as the case may be, the hollow fiber membrane, can also be designed as a porous or microporous membrane.

Thus, other membranes which are suitable to be employed with medical products and which are already used in the state of the art, can be employed with the gas exchange membrane, example given, membranes used in connection with hemofiltration or dialysis.

The object of the gas exchange membrane is, on the one hand, to serve as a matrix for the colonization with endothelial cells and epithelial cells, further permitting supply of the epithelial cells from the direction of the blood side. Therefore, the matrix is configured in such a way that it does not impede the physiological interactions of blood-side cells (endothelium) and air-side cells (pneumocytes), while forming a mechanical framework for these cells.

Initial studies have shown that the already clinically approved gas exchange membrane made from polymethyl-pentene can be colonized with pneumocytes, in order to convert the foreign surface into a biological surface structure. The pneumocytes used were generated by inducing the in vitro expression of endodermal phenotype in human CD34⁺ haematopoietic stem cells (HSC) that were obtained from umbilical cord blood. By cultivation in the presence of growth factor activin A, the HSC were able to be differentiated to the phenotype of distal lung epithelium.

The colonization of the blood side with endothelial cells can be carried out using current techniques, see for example XU C. et al.: IN VITRO STUDY OF HUMAN VASCULAR ENDOTHELIAL CELL FUNCTION ON MATERIALS WITH VARIOUS SURFACE ROUGHNESS, IN J BIOMED. MATER. RES. A. (2004 OCT) 1; 71(1):154-161.

The authors describe how human vascular endothelial cells can be cultured on pretreated, three-dimensional support structures that can serve to replace blood vessels of small diameters.

Bos et al.: BLOOD COMPATIBILITY OF SURFACES WITH IMMOBILIZED ALBUMIN-HEPARIN CONJUGATE AND EFFECT OF ENDOTHELIAL CELL SEEDING ON PLATELET ADHESION, in: J BIOMED. MATER. RES. (1999 DEC) 5; 47(3):279-291, describe the colonization of an albumin-heparin coating with endothelial cells that adhere to the coating and proliferate on it.

When using polymethyl-penten membranes, it is a further advantage that these membranes represent a layer being leak tight for plasma when used in connection with a endothelial coating only. The membranes disclosed in the state of the art and coated with endothelial cells have the disadvantage, that they allow plasma leakage to the other side of the membrane, or that they have to be coated in a complex way to avoid plasma leakage. On the other hand, the PMP membranes are advantageously plasma leak tight.

In a further embodiment of the artificial lung system according to the invention the gas exchange membrane can be provide with a coating of additional substances, which affect adhesion and/or differentiation of the cells. Such factors are, example given, components of the extracellular matrix (ECM), as for example fibronectin, laminin, tenascin und vitronectin, oder growth factors, for example EGF (epidermal growth factor), FGF (fibroblast growth factor), GCSF, GGF (glia growth factor), GMCSF, GMA, GMF (glia maturation factor), IGF (insulin like growth factor), interferones, interleukines, lymphokines, MCSF, monokines, NGF (Nerve growth factor), NO (nitrogen mono oxide), PD-ECGF (platelet derived endothelial cell growth factor), PDGF (platelet derived growth factor), TGF (transforming growth factor), TNF (tumor-necrosis-factor), or, on the other hand, antibodies, nucleic acids, apatamers, etc., which either affect adhesion and/or differentiation of cells.

Further advantages will become clear from the description and from the attached drawing.

It will be appreciated that the aforementioned features and those still to be explained below can be used not only in the respectively cited combinations but also in other combinations or singly, without departing from the scope of the present invention.

### BRIEF DESCRIPTION OF THE DRAWINGS

An illustrative embodiment of the invention is shown in the drawing and is explained in more detail in the following description.

### DETAILED DESCRIPTION OF PREFERRED EMBODIMENTS

The single drawing shows the novel artificial lung system that can be used both to produce an extracorporeal or implantable lung assist system and also to produce a lung model system for the examination of airway stresses.

In the single FIGURE, reference number 10 designates an artificial lung system, shown extremely schematically in the FIGURE.

The artificial lung system 10 comprises a gas exchange membrane 11 that separates a blood side 12 from an air side 14. The gas exchange membrane 11 faces with its foreign surfaces 15 and 16 into a closed blood chamber 17 on the blood side 12 and into a closed air chamber 18 on the air side 14.

In the context of the present invention, “foreign surface” is understood as an artificial surface which, per se, does not have to be biocompatible.

In the present case, the gas exchange membrane 11 is a diffusion membrane made from polymethylpentene (PMP), as is used in the NovaLung iLA lung assist system. Such PMP membranes can be obtained, for example, from the company Membrana, Oehder Str. 28, D-42289 Wuppertal, Germany, under the name Oxyplus capillary membrane (order No. PMP 90/200).

The gas exchange membrane 11 is a membrane made up of interwoven hollow fibres, the outside of the hollow fibres facing towards the blood side 12, and the inside of the hollow fibres facing towards the air side 14. This geometric configuration is not shown in the figure. Instead, the gas exchange membrane 11 is only indicated schematically.

The foreign surface 16 in the air chamber 18 is colonized with epithelial cells 21. It is, as it were, completely covered by an alveolar cell or pneumocyte lawn.

The foreign surface 15 in the blood chamber 17 is colonized with endothelial cells 22. It is, as it were, completely covered by a lawn of endothelial cells.

The blood chamber 17 contains blood baffle plates 23 which ensure that, between a venous attachment 24 and an arterial attachment 25, a homogeneous blood flow 26 is generated that ensures a uniform perfusion of the endothelial cells 22.

The air chamber 18 is connected to an air inlet 27 and to an air outlet 28, between which an air stream 29 can be generated for ventilation of the pneumocytes 21.

The air outlet 28 can be connected, for example, to an underpressure system 31, such that the ventilation of the air chamber 18 takes place physiologically, that is to say with underpressure. On the other hand, the air outlet 28 can also be linked to the lungs, in which case the air inlet 27 is then linked to the trachea. The underpressure system 31 can also be a pressure chamber 31 that generates an oscillating air stream.

Finally, it is also possible to connect the air inlet 27 to a connector piece 30 that comprises an aeration inlet 32, a foreign substances inlet 33 and a humidifying inlet 34.

The artificial lung system 10 can now be used, for example, to produce an extracorporeal lung assist system.

For this purpose, the air outlet 28 is connected to the underpressure system 31, and the air inlet 27 is connected to a foreign substances filter (not shown in the FIGURE) via which the aspirated air can also be humidified.

The venous attachment 24 is connected to a vein of the patient via a percutaneous cannula, for example, while the arterial attachment 25 is connected, likewise via a percutaneous cannula, to an artery of the patient. The artery and vein used can be, for example, the subclavian artery and subclavian vein, to which the artificial lung system is subcutaneously attached via vascular prostheses.

In this way, the blood flow 26 is moved by the patient's heart, such that no additional mechanical pump is needed. On the air side, ventilation takes place with underpressure, such that the air chamber 18 is ventilated physiologically.

Since the membrane 11 is now colonized on the air side 14 with pneumocytes 21 and on the blood side 12 with endothelial cells 22, the artificial lung 10 simulates as it were the physiological situation, with the pneumocytes 21 being physiologically ventilated and the endothelial cells 22 being perfused with, for example, the patient's blood, with the result that optimal growth conditions and functional conditions prevail.

The artificial lung system now ensures oxygenation of the blood flow 26, with oxygen thus passing from the air chamber 18 into the blood chamber 17. At the same time, carbon dioxide is withdrawn from the blood stream 26, with CO₂ thus passing from the blood chamber 17 into the air chamber 18.

The inventors of the present application have discovered that an extracorporeal lung assist system of this kind can be used over long periods of time, because the endothelial cells 22 and the physiological flow conditions in the blood chamber 17, further supported by the blood baffle plate 23, prevent any irritation of the flowing blood, such that the neointima formation, coagulation activation and inflammatory reactions, etc., observed in the prior art, no longer occur.

Since, in addition to this, pneumocytes 21 cover the foreign surface 16 on the air side 14, a biological defense takes place there, with the pneumocytes forming the physiological barrier with respect to the individual patient. The pneumocytes are supplied with nutrients through the blood flow 26, that is to say through the gas exchange membrane 11. For this purpose, it is necessary to provide the gas exchange membrane 11 with a sufficient pore size and geometry in order, on the one hand, to permit exchange of nutrients and intercellular communication while, on the other hand, preventing the passage of blood into the air chamber 18.

Alternatively, the artificial lung system 10 can also be used to produce an implantable lung assist system. In this case, the venous attachment 24 and the arterial attachment 25 are connected with suitable cannulas to veins and arteries inside the patient's body. The air inlet 27 is linked inside the body to the trachea, and the air outlet is connected, for example, to an implanted pressure chamber 31 which is alternately expanded and compressed, either via an external mechanical energy source or via endogenous muscles. In this case, therefore, only one attachment to the airway system is made and an oscillating air stream is generated that supplies the biohybrid lung in a natural manner with ventilation gas in bidirectional flow. The ventilation of the air chamber 18 thus takes place via the breathing activity of the patient's lungs, and the perfusion of the blood chamber 17 takes place via the patient's heart activity, both therefore taking place physiologically.

In an implantable lung assist system, the air inlet 27 is therefore used to attach the closed air chamber 18 to the natural airways of the patient.

On the other hand, the artificial lung system 10 can also be used to produce a lung model system for the examination of airway stresses or of pulmotoxic substances in the perfusate/blood stream. In this case, the blood side is connected via the venous attachment 24 and the arterial attachment 25 to an artificial perfusion system that generates an artificial blood circulation via which the blood chamber 17 is perfused physiologically in a pulsatile manner.

The pulmonary air outlet 28 is connected to the underpressure system 31, and the tracheal air inlet 27 is connected to the connector piece 30, such that the ventilation of the air chamber 18 likewise takes place physiologically, that is to say with underpressure. The pneumocytes 21 and the endothelial cells 22 therefore grow and live as before under physiological conditions.

Volatile substances or foreign gases can be introduced into the air stream 29 via the foreign substances inlet 33, such that the effect of these foreign substances on the pneumocytes 21 and the endothelial cells 22 can be examined in the context of toxicology studies. This lung model system can therefore replace the animal model systems that have hitherto been used, for example in order to determine the toxicity or maximum workplace concentration of certain substances.

It should also be noted that, for all three of the applications just described, it is not absolutely necessary for both foreign surfaces 15 and 16 to be colonized with biological cells. An appreciable advantage in all three applications is already achieved when the foreign surface 15 on the blood side 12 is colonized with endothelial cells 22.

Such colonization can be achieved, for example, in the manner described in the aforementioned articles by Bos et al. and by Xu et al., the content of which is, by this reference, made part of the subject matter of the present application.

In this case, the foreign surface 16 on the air side 14 can be provided with a nonvital coating, for example with protein and heparin.

On the other hand, it is also possible to only colonize the foreign surface 16 on the air side 14 with pneumocytes and to provide the foreign surface 15 on the blood side 12 with a nonvital coating. The latter may be expedient particularly if, in the context of a lung model system, the blood chamber 17 is permeated by artificial blood or a medium that is simply used to supply nutrients to the pneumocytes on the air side 4.

A first pilot study has shown that pneumocyte colonization of the gas exchange membrane 11 made from PMP is possible.

For this purpose, the human type II pneumocyte tumour cell line A549 (American Type Culture Collection, Virginia, USA, # CCL 183; Lieber et al., Int. J. Cancer (1976) 17:62-70) and the murine SV40-transformed type II pneumocyte cell line MLE 12 (American Type Culture Collection, Virginia, USA, # CRL 2110; Wikenheiser et al., PNAS USA (1993) 90:11029-11033) were cultivated with 10% (v/v) fetal calf serum in Dulbecco's modified Eagle's medium (DMEM, Invitrogen, Paisley, UK).

Small pieces of the PMP fibre membrane measuring approximately 1 cm×0.5 cm were cut out, sterilized with UV radiation and placed in the culture medium. The pneumocytes were seeded out onto the surface of the fibres and incubated at 37 degrees C.

The cell growth was able to be observed with an inversion microscope. Within a few days, the cells had spread out across the surface of the hollow fibres and, within two weeks, they were also growing inside the fibres.

These first experiments show that it is possible to colonize the inner surfaces of hollow fibres with pneumocytes.

It is not absolutely essential to use human (A549) or murine (MLE 12) cell lines as the source of the pneumocytes. Instead, the pneumocytes can also be cultivated by differentiation of human CD34⁺ haematopoietic stem cells (HSC) that are obtained from umbilical cord blood; see Albera et al.: “Human CD34⁺ Haematopoietic Stem Cells (HSC) From Umbilical Cord Blood Display An Endodermal Phenotype When Exposed To Activin A In Vitro”, Blood (2005) 106:484 A.

In these HSC, it was possible to induce the in vitro expression of endodermal phenotype by culturing in the presence of the growth factor activin A; the HSC were therefore able to be differentiated to the phenotype of the distal lung epithelium.

Although the abovementioned colonization tests were carried out using robust pneumocyte cell lines, initial results achieved by the Applicant show that pneumocytes which were cultivated from umbilical cord blood, and which differentiated in the presence of growth factor activin A to the phenotype of distal lung epithelium, could also be used for colonization on the air side of PMP gas exchange membranes.

Therefore, what is claimed, is:

1. Artificial lung system, with a gas exchange membrane that separates a blood side from an air side, the gas exchange membrane comprising a foreign surface on the blood side and a foreign surface on the air side, wherein the foreign surface on the blood side and/or the foreign surface on the air side is colonized with biological cells.

2. Artificial lung system according to claim 1, wherein the foreign surface is colonized completely with biological cells.

3. Artificial lung system according to claim 1, wherein the foreign surface on the blood side is colonized with endothelial cells.

4. Artificial lung system according to claim 1, wherein the foreign surface on the air side is colonized with alveolar epithelial cells.

5. Artificial lung system according to claim 1, wherein the foreign surface not colonized with biological cells is provided with a nonvital coating.

6. Artificial lung system according to claim 1, wherein the gas exchange membrane is a diffusion membrane.

7. Artificial lung system according to claim 6, wherein the gas exchange membrane between endothelium and pneumocytes is produced from polymethylpentene.

8. Artificial lung system according to claim 1, wherein the gas exchange membrane is porous or microporous.

9. Artificial lung system according to claim 1, wherein the blood side comprises a closed blood chamber with inlet and outlet ports for attachment to a natural blood circulation or to an artificial perfusion system.

10. Artificial lung system according claim 1, wherein the air side comprises a closed air chamber with inlet ports for attachment to natural airways or to an artificial ventilation system.

11. Artificial lung system according to claim 10, wherein the artificial ventilation system has an inlet for foreign substances.

12. Artificial lung system according to claim 1, wherein the biological cells are autologous cells that are preferably obtained from the respiratory tract, a segment of a superficial cutaneous vein, or the umbilical cord.

13. Method for producing an extracorporeal lung assist system, comprising the step of employing the artificial lung system according to claim 1.

14. Method for producing an implantable lung assist system, comprising the step of employing the artificial lung system according to claim 1.

15. Method according to claim 14, wherein an inlet port of the air chamber of the artificial lung system is designed for attachment to natural airways, and the other inlet port of the air chamber is connected to a pressure chamber that generates an oscillating air stream in the air chamber by means of alternating expansion and compression.

16. Method for producing a lung model system for the examination of airway stresses, comprising the step of employing the artificial lung system according to claim 1.
